(19) United States
(12) Patent Application Publication
(10) Pub. No.: US 2022/0276216 A1
(43) Pub. Date: Sep. 1, 2022

(54) METHODS AND SYSTEMS FOR EVALUATING ORGANIC CONTAMINANTS IN WATER

(71) Applicant: CHEMTREAT, INC., Glen Allen, VA (US)

(72) Inventors: Megan PETTYGROVE, Quinton, VA (US); Mark PUCHOVICH, Henrico, VA (US); James WILKINS, Midlothian, VA (US); Benjamin NIEMASECK, Chesterfield, VA (US); William HENDERSON, Richmond, VA (US); Brian SILVA, Morrow, OH (US)

(73) Assignee: CHEMTREAT, INC., Glen Allen, VA (US)

(21) Appl. No.: 17/673,987

(22) Filed: Feb. 17, 2022

Related U.S. Application Data
(60) Provisional application No. 63/154,225, filed on Feb. 26, 2021.

(51) Int. Cl. G01N 33/18; G01N 21/64; C02F 1/58

(52) U.S. Cl. CPC G01N 33/18; G01N 21/64; C02F 1/58; C02F 2209/04; C02F 2209/05; C02F 2209/06; C02F 2209/20

(57) ABSTRACT

Methods and systems are described for evaluating the level of organic contaminants in water, and in particular water that is used as boiler feedwater in food processing facilities such as sugar factories. The method includes measuring at least one parameter of the water including pH, conductivity, and/or total organic carbon, and, based on the measured values, determining whether corrective action needs to be taken to reduce the levels of organic contaminants.

### CROSS-REFERENCE TO RELATED APPLICATIONS

This application claims the filing date benefit of U.S. Provisional Application No. 63/154,225 filed on Feb. 26, 2021, the entirety of which is incorporated by reference herein.

### TECHNICAL FIELD

This disclosure relates generally to systems and methods that are able to detect and evaluate organic contaminants in water, and more particularly relates to detection of such contaminants in water that is used as boiler feedwater. As one example, such contamination can occur in the condensate water from evaporation stages used in sugar production. Embodiments of the invention are described in connection with sugar production processes but it would be understood that the invention could be applied to other production processes that experience organic contaminants in water.

### BACKGROUND

A typical sugar production process is shown in FIGS. 1A and 1B. FIG. 1A illustrates the flume system, in which the vegetables are introduced and washed with water before being further processed, e.g., to produce sugar. The flume wash typically includes a stream of water that transports the beets while washing them. The stream normally terminates at a beet washer tank where agitation and a series of beater bars remove dirt from the beets. The beets are typically then conveyed onto a spray table where the beets are sprayed with water prior to being sent to the slicer. The flume system is a closed system with primary water loss from water on the beets that is carried into the process stages. FIG. 1B illustrates the sugar production stages that are downstream of the flume system. Once the beets arrive they are sent to the slicer where they are sliced into cossettes that may resemble either ruffled potato chips, or shoestring potatoes depending on beet quality at the time. From the slicer they are sent to a diffuser to extract the sugar. After the diffusers, the water contains solid particles, dissolved sugars and dissolved non-sugars. The sugar content is around 14-18% in solution and 85-92% purity. In order to remove the non-sugars, such as lignin or tannin, lime is added to raise the pH to around 11-12 which helps facilitate coagulation of particulates and non-sugars. After the first lime addition, the juice is heated and more lime is added to react any non-sugars that remain dissolved. At the carbonation stages, the pH is dropped to 9.8-10.5 by adding CO₂ to help solids precipitate. From the Dorr or clarifier overflow after 1^{st} carbonation, the juice is sent to a 2^{nd} carbonation step and subsequent steps, as needed. After filtration, the juice is referred to as “thin juice”. It is a light amber color and is typically around 14-18% sugar in solution at around 88-92% purity. Thin juice goes through five to seven evaporator stages, which concentrate the juice into “thick” juice. The “thick juice” is high in dissolved sugar at around 60-65%. Thick juice and a mixture of syrup returns from the spinners, are blended in the standard liquor tank, filtered, and sent to the vacuum pan to crystallize into white sugar. That portion of the syrup that can no longer be crystallized into sugar is sent to the molasses tanks. Separators or MD (molasses desugarization) processes may help remove sugar from the molasses with remaining liquor being used for animal feed or dust control. Cane molasses is marketed for use by consumers or consumer products.

As shown in more detail in FIG. 2, in the process of converting thin juice to thick juice, sugar laden water goes through a series of evaporators to concentrate up the sugar content in the process fluid. The condensate from this evaporation process can be used as the boiler feedwater, together with make up water as needed. Water coming from vegetable washing or extraction stages can also be used as boiler feedwater. Occasionally, there are upsets which cause sugar, and other contaminants in the fluid to carry over into the condensate. These organics are detrimental to the operation of boilers in many ways, including pH depression, corrosion, and fouling.

Currently, sugar production facilities detect the presence of contaminants in the water that is used as boiler feedwater solely by measuring the fluorescence, tuned to wavelengths that are considered to reflect the excitation/emission (ex/em) wavelengths of thin juice contaminants (365 nm/470 nm). Thus, conventional methods associate an increase in the intensity of these fluorescent signals to increased organic contamination of the water, and thus can take corrective action when spikes in such fluorescence is observed.

### SUMMARY

In connection with this disclosure, the inventors have discovered that this known method for detecting organic contaminants has several drawbacks. First, as the contaminants move through the evaporators, they break down and form products whose ex/em maxima is significantly different from the parent compounds. This results in decreased sensitivity of contaminant carryover. Second, the optimal ex/em maxima to detect contaminants may vary because the quality of the beets or sugar cane can change throughout the course of a campaign or season, e.g., based on when the source is harvested, how long it is stored before processing, and the environmental conditions of any such storage. For example, sugar beets are commonly stored before slicing. This time spent out of the ground in storage can cause beets to rot and begin germination, and lignins and non-sugars are usually higher for aged beets. Finally, sugar itself is not fluorescent, and thus existing measures do not detect contamination from sugar. Accordingly, there is a need for methods that are able to more accurately and reliably detect or predict levels of organic contaminants that are present in boiler feedwater.

According to one aspect, this disclosure provides a method for evaluating water that is used as boiler feedwater. The method includes measuring at least one parameter of the water that includes pH, conductivity, and/or total organic carbon (TOC), and based on the at least one measured parameter, determining whether to take corrective action to reduce the amount of organic contaminants in the water and/or mitigate effects of the organic contaminants in the water.

According to another aspect, this disclosure provides an apparatus for evaluating water that is used as boiler feedwater in a food processing facility. The apparatus includes a processor that is programmed to (i) receive a signal corresponding to a measured parameter of the water that includes at least pH, conductivity, total organic carbon (TOC), and/or oxidation reduction potential (ORP); and (ii) based on the received signal corresponding to the measured parameter, generating a signal to control at least one operating parameter of the food processing facility and/or generating a signal that causes a display to display an alert.

According to another aspect, this disclosure provides a method for controlling an amount of an organic contaminant in boiler feedwater that is used in a boiler of an evaporator stage in a sugar processing facility. The method includes (i) measuring at least one parameter of the boiler feedwater that is selected from the group consisting of pH, conductivity, total organic carbon (TOC), and oxidation reduction potential (ORP); and (ii) based on the at least one measured parameter, taking at least one corrective action to reduce an amount of organic contaminant in the boiler feedwater and/or mitigate effects of the organic contaminant in the boiler feedwater.

### BRIEF DESCRIPTION OF THE DRAWINGS

FIG. 1A is a schematic diagram of a washing water system in a beet sugar factory;

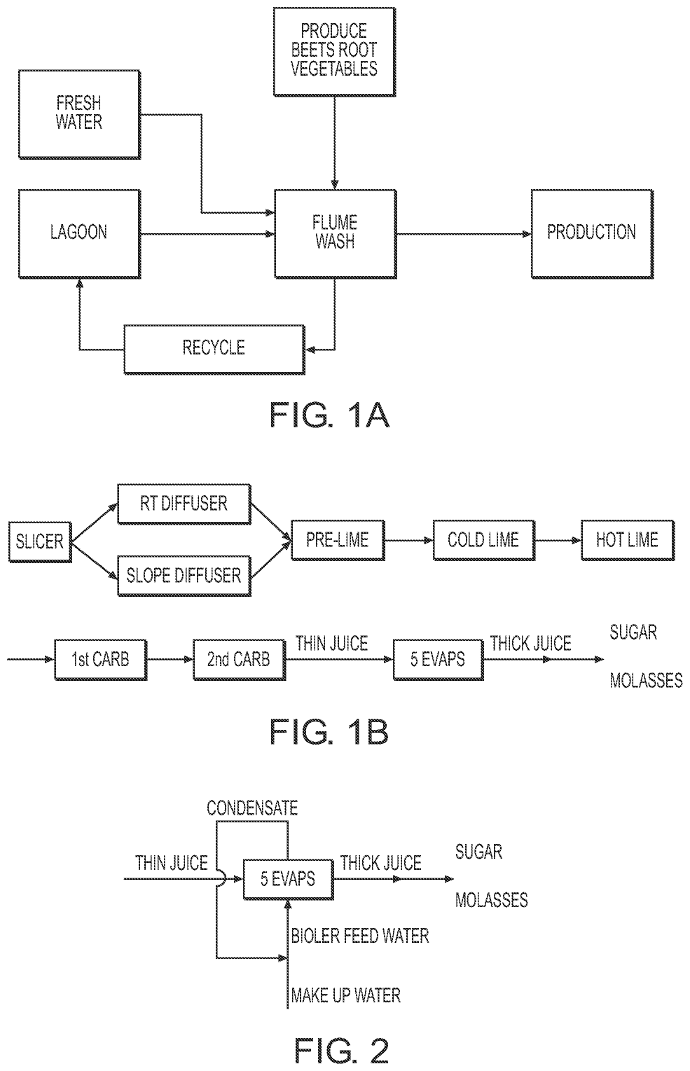

FIG. 1B is a schematic diagram of a typical beet sugar processing system;

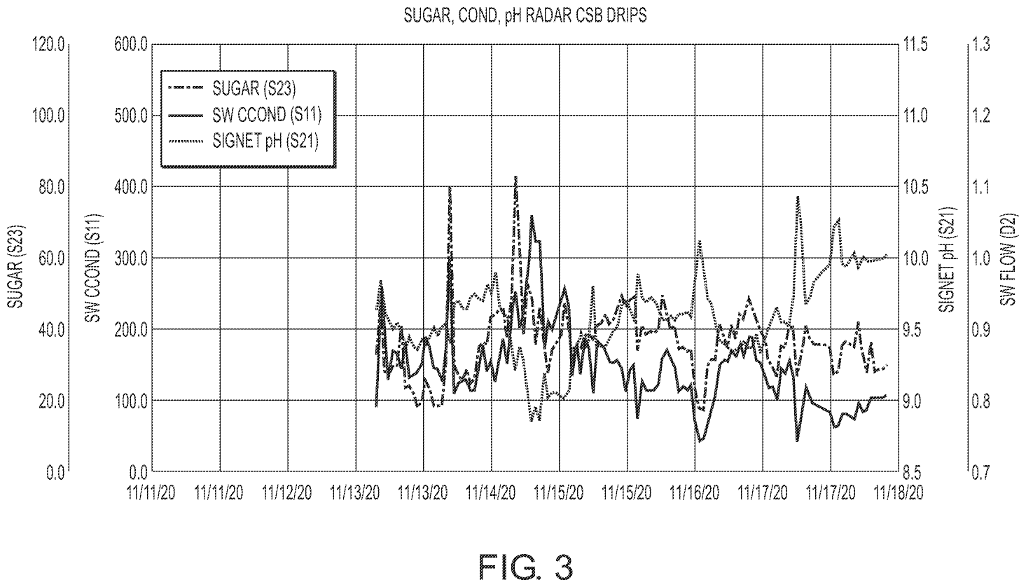

FIG. 2 is a schematic diagram illustrating the evaporation stage in a typical beet sugar processing system;

FIG. 3 is a graph showing measured values of fluorescence, conductivity, and pH for a condensate stream of an evaporator in a beet sugar factory;

FIG. 4 is a graph showing measured values of flow rate, fluorescence, conductivity, and pH for a boiler feedwater stream to an evaporator in a beet sugar factory;

FIG. 5 is another graph showing measured values of fluorescence, conductivity, and pH for a condensate stream of an evaporator in a beet sugar factory;

FIG. 6 is a graph showing the correlations between three different fluorescence wavelengths and the concentrations of lignin and tannin in samples from sugar beet factories;

FIG. 7 is a graph showing the correlations between four different fluorescence wavelengths and the concentrations of sucrose in the thin juice from a sugar beet factory;

FIG. 8 is a graph showing the correlation between sucrose concentration and lignin/tannin concentration in the thin juice from a sugar beet factory;

FIG. 9 is a graph showing the correlation between betaine concentration and conductivity in the thin juice from a sugar beet factory;

FIG. 10 is a graph showing the correlations between four different fluorescence wavelengths and the concentrations of lignins/tannins in a first evaporator condensate stream from a sugar beet factory;

FIG. 11 is a graph showing the correlations between four different fluorescence wavelengths and the concentrations of lignins/tannins in a second evaporator condensate stream from a sugar beet factory;

FIG. 12 is a graph showing the correlations between four different fluorescence wavelengths and the concentrations of lignins/tannins in boiler feed water from a sugar beet factory; and

FIGS. 13A-13C are graphs shown the measured fluorescence intensity at various stages in a sugar beet processing facility over the course of a season.

### DETAILED DESCRIPTION OF EMBODIMENTS

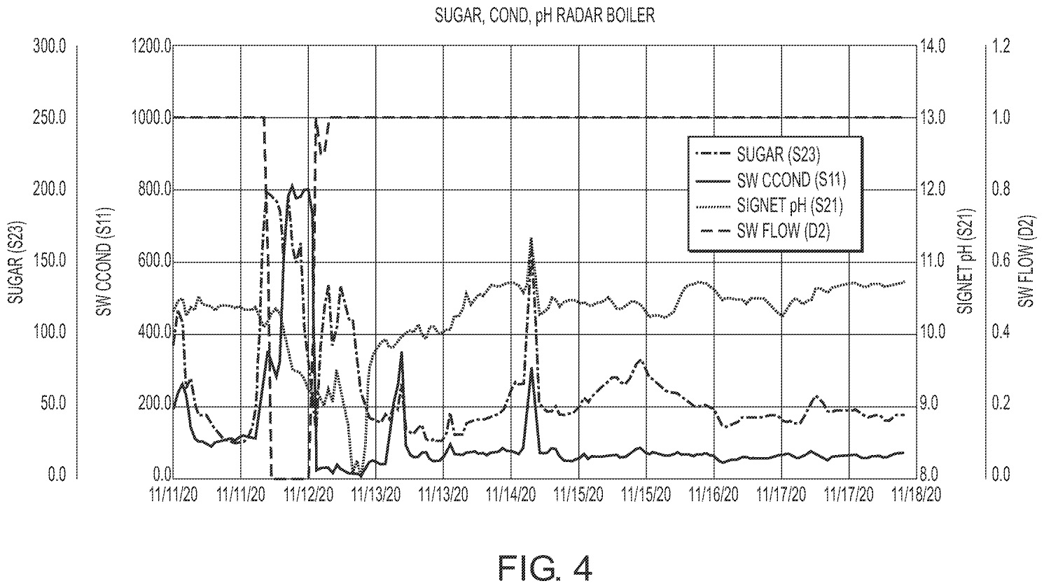

Disclosed embodiments include methods for determining contamination of boiler feedwater in food production facilities such as sugar factories. As indicated in connection with FIG. 2 the boiler feedwater may, in part or in whole, come from condensate from the evaporation process, or it may include water that is used in the washing or extraction stages. These water sources can have organic contaminants that can negatively affect the boiler, including sugars, polymeric carbohydrates, lignins, tannins, organic acids (e.g., betaine), and/or breakdown products of these components. For example, in the case of evaporator condensate, the contaminants may become entrained in the condensate based on leaks, boil-over entering the condenser, and possibly contaminants sublimating in the condenser.

In one aspect, it has been discovered that the presence of organic contaminants can be accurately detected based on pH, total organic carbon (TOC), and/or conductivity of the water. In another aspect, it was discovered that organic contaminants in water can be detected using optimized fluorescence wavelengths, e.g., that are based on breakdown components from parent contaminants. Thus, it has been discovered that changes in the pH, conductivity, TOC and/or combinations thereof can be correlated with contaminants (including, e.g., sugars, lignins/tannins, betaine and other modified amino acids, and breakdown products) in the water. Fluorescence intensity at optimized wavelengths can likewise be correlated with certain contaminants in the water. Accordingly, each of these detection techniques may be used alone or in combination with each other (i.e., two or more parameters) to more accurately and reliably measure the organic contaminants and to allow for predictive modeling of upcoming plant upsets.

The pH, conductivity, and fluorescence measurements can be taken with probes that are positioned to measure the condensate from one or more of the evaporators, e.g., the first drip from the first evaporation stage, or the second drip from the second evaporation stage, etc., or are positioned to directly measure the boiler feedwater. Probes that are positioned to directly measure the boiler feedwater can be located at the feedwater off of the first evaporator(s), off the of the second evaporator(s), or off of the raffinate evaporator, for example. The location can be determined by plant piping configurations and/or likely locations of possible contaminants.

The TOC is a laboratory measurement, and can be performed on water samples taken from a condenser drip or from the boiler feedwater, for example. The TOC can be correlated with other parameters that can be measured in real time, such as the pH, fluorescence, or oxidation reduction potential (ORP), for example.

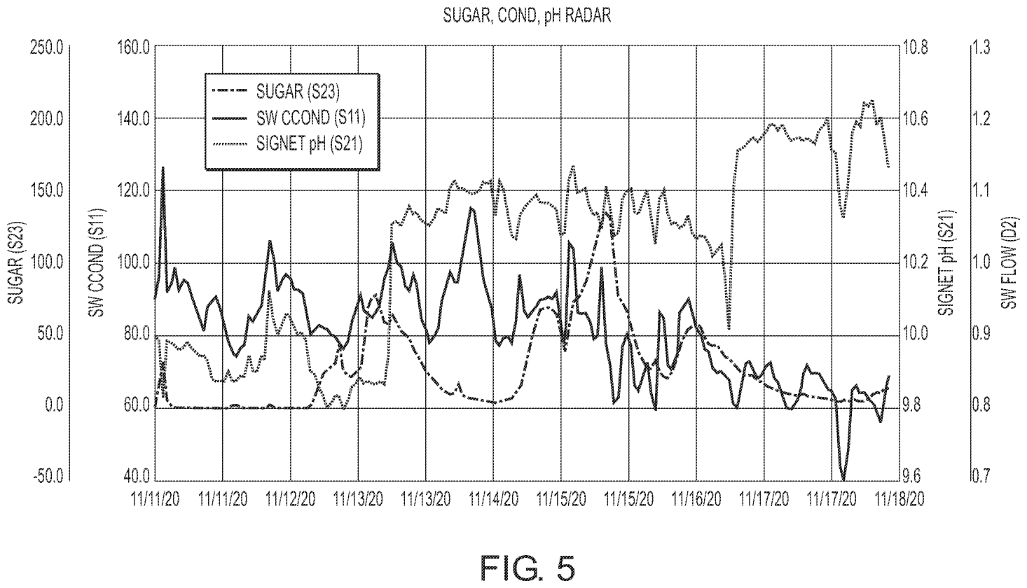

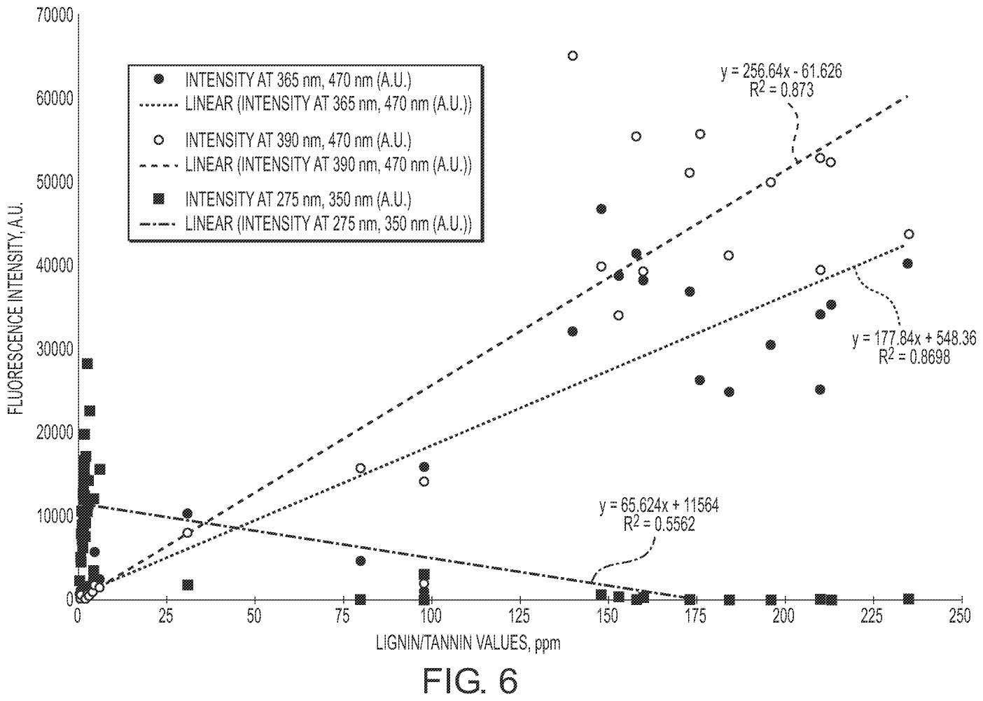

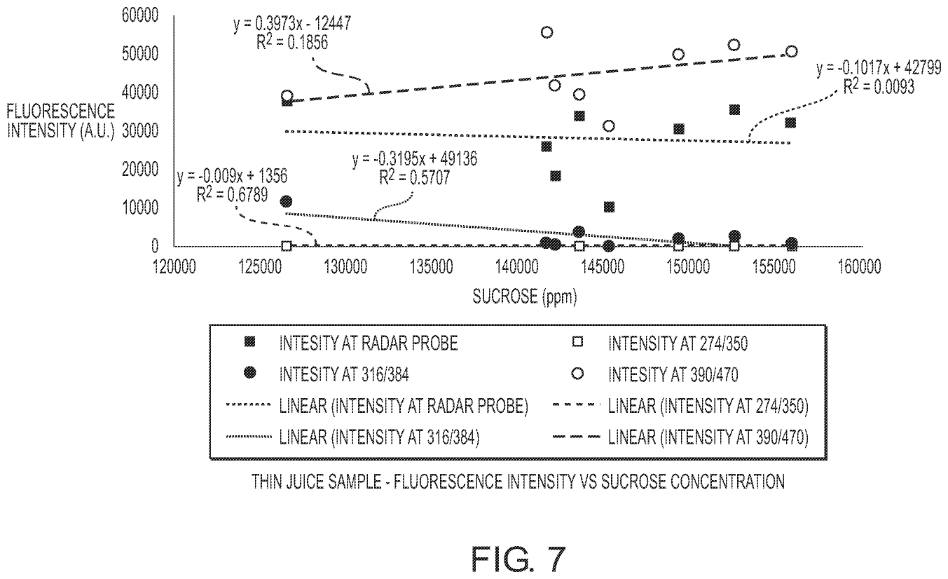

FIG. 3 is a graph showing real-time measured values of fluorescence, conductivity, and pH for a condensate stream of an evaporator in a beet sugar factory over five days. The fluorescence is measured at ex/em of 365 nm/470 nm. As can be seen in FIG. 3, in several instances (e.g., 11/16/20 and 11/17/20) abrupt changes in the pH, fluorescence intensity, and conductivity coincide. In this case, the fluorescence intensity and conductivity change inversely to the pH. FIG. 4 shows similar real-time measured values of the boiler feedwater over 7 days, and FIG. 5 shows real-time measured values of another condensate stream for 7 days. In each trial, abrupt changes in two or more of the parameters coincide (e.g., 11/14/200 in FIG. 4, and 11/11/20 in FIG. 5). In some cases, the changes in a parameter can be proportional to other parameters (e.g., FIG. 4), and in some cases the changes can be inversely proportional (e.g., FIG. 3). In either case, it is believed that such abrupt changes correspond to upsets in the water, such as spikes in the contaminants level. The use of pH and/or conductivity values, in addition or as an alternative to fluorescence measurements, can therefore provide reliable indications of contamination events, and can allow detection of contamination events that standard fluorescence detection could miss.

As indicated above, conventional fluorescence measurements in this field to detect organic contaminants at an ex/em of 365 nm/470 nm. Applicant's copending U.S. patent application Ser. No. 16/622,369, the entirety of which is incorporated by reference herein, describes some suitable fluorescence parameters for detecting lignins and tannins, as well as additional contaminants which are likely breakdown products of lignins and tannins. A fluorescence probe can be used to detect lignins and tannins at excitation wavelengths from 380-400 nm, preferably around 390 nm, and emission wavelengths at 460-480 nm, preferably around 470 nm. Another fluorescence probe can be used to detect likely breakdown products of tannins and lignin at excitation wavelengths in a range of from 260-290 nm, preferably around 275 nm, and emission wavelengths at around 340-360 nm, preferably around 350 nm.

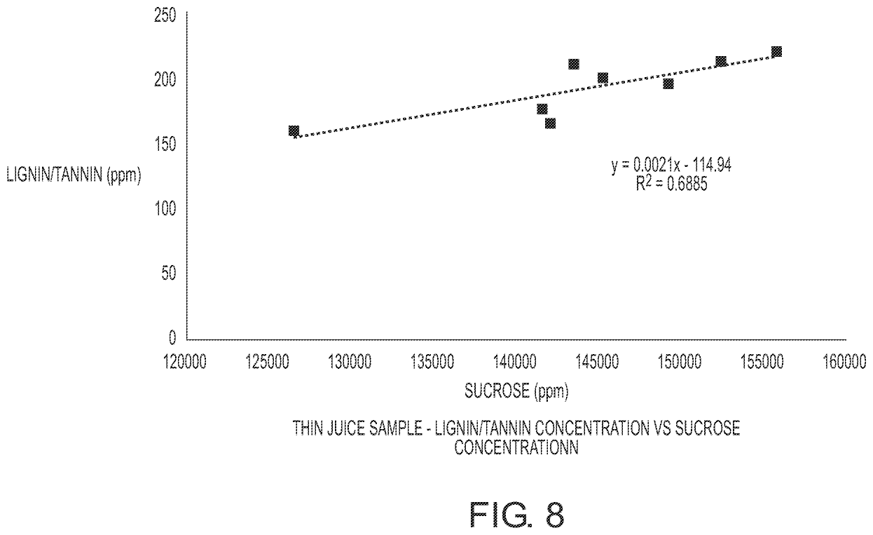

FIG. 6 is a graph showing the correlations of three different fluorescence ex/em wavelengths (wavelengths 365 nm/470 nm; wavelengths 390 nm/470 nm; wavelengths 275 nm/350 nm) to the concentrations of lignin and tannin in thin juice samples and condensate samples from several U.S. sugar beet factories. The data shows that there is a good correlation between the concentrations of these contaminants and fluorescence intensity at both 365/470 (as has been conventionally used), and at 390/470. As can be seen, the correlation at 390/470 is somewhat better than 365/470 and may be more sensitive than these conventional wavelengths. The correlation at 275/350 is inversely proportional to the lignin/tannin concentration, and it is believed that the 275/350 fluorescence detects breakdown products of lignins and tannins.

Table 1 below shows raw data of water in several U.S. sugar beet factories at various stages of the production process. The data includes fluorescence measurements at various wavelengths, measured lignin and tannin concentrations, TOC, chemical oxygen demand (COD), pH and conductivity. Table 2 shows similar raw data for water in several sugar cane plants in the United States and Latin America.

TABLE 1

| | | | | Intensity | Intensity | Intensity |
|---|---|---|---|---|---|---|
| | Max | Max | Relative | at 275 nm, | at 365 nm, | at 390 nm, |
| | Excitation | Emission | Intensity | 350 nm | 470 nm | 470 nm |
| Sample Type | (nm) | (nm) | (A.U.) | (A.U.) | (A.U.) | (A.U.) |
| Pond 3 Inlet | 344 | 430 | 13853 | 1730 | 10305 | 7983 |
| Condenser | 284 | 330 | 21751 | 15532 | 2461 | 1487 |
| Clarifier Underflow | 404 | 494 | 2609 | 20 | 1013 | 1924 |
| Clarifier Underflow | 350 | 444 | 17541 | 2931 | 15853 | 14156 |
| Filtered | | | | | | |
| Raffinate 100× dilution | 400 | 478 | 18601 | 2 | 4560 | 15694 |
| Condensate | 264 | 338 | 25476 | 22583 | 1658 | 568 |
| Thick Juice | 514 | 576 | 18248 | 302 | 40 | 45 |
| Condenser to Boiler | 274 | 334 | 2350 | 2217 | 772 | 536 |
| Thin Juice | 394 | 468 | 65901 | −8 | 31996 | 64930 |
| Thin Juice Run #2 | 394 | 468 | 65901 | −8 | 31996 | 64930 |
| Condensate | 316 | 384 | 28721 | 10715 | 779 | 261 |
| Condensate Run #2 | 316 | 384 | 28721 | 10715 | 779 | 261 |
| Thin Juice | 356 | 440 | 46571 | 359 | 38664 | 33908 |
| Condensate | 320 | 384 | 76781 | 12119 | 2404 | 968 |
| Waste Collection Tank | 268 | 330 | 6289 | 4537 | 1030 | 666 |
| 1st Condensate | 274 | 340 | 9672 | 8941 | 767 | 206 |
| 2nd Condensate | 262 | 338 | 13805 | 8923 | 309 | 112 |
| CSB drips | 318 | 394 | 12512 | 11290 | 1509 | 392 |
| Raff Drips | 322 | 386 | 177877 | 3451 | 5717 | 1736 |
| Thin Juice | 362 | 444 | 54660 | 571 | 46625 | 39765 |
| Thin Juice | 386 | 464 | 52593 | 17 | 36747 | 50933 |
| First Evap Stage | 264 | 336 | 20570 | 11975 | 268 | 126 |
| 2nd Drips | | | | | | |
| Second Evap Stage | 314 | 384 | 12512 | 9290 | 327 | 131 |
| 2nd Drips | | | | | | |
| Thin Juice | 374 | 454 | 44055 | 280 | 38050 | 39173 |
| First Evap Stage | 262 | 336 | 45167 | 19733 | 384 | 193 |
| 2nd Drips | | | | | | |
| Second Evap Stage | 262 | 336 | 28302 | 15113 | 240 | 95 |
| 2nd Drips | | | | | | |
| Radar Panel | 262 | 334 | 31440 | 15878 | 302 | 126 |
| (Condensate | | | | | | |
| composite) | | | | | | |
| 1st Condensate | 270 | 334 | 11595 | 10279 | 714 | 300 |
| 2nd Condensate | 262 | 334 | 41168 | 14436 | 610 | 296 |
| CSB drips | 316 | 390 | 15339 | 11490 | 1613 | 490 |
| Raff Drips | 274 | 336 | 13495 | 12191 | 780 | 229 |
| Thin Juice | 394 | 466 | 53943 | −4 | 25071 | 52716 |
| Evap 19:50? | 268 | 336 | 10701 | 9361 | 336 | 124 |
| 2nd Evap 9:55 | 262 | 336 | 27008 | 12481 | 373 | 155 |
| CSB drips 9:40 | 274 | 340 | 18232 | 17172 | 1215 | 536 |
| Raff Drips 9:35 | 274 | 336 | 8709 | 7602 | 296 | 114 |
| Thin Juice 9:30? | 384 | 464 | 56743 | 21 | 41302 | 55303 |
| Thin Juice Softened, | 388 | 468 | 42696 | 4 | 35184 | 52186 |
| Sulfer, Caustic 1:53 | | | | | | |
| First Evap Stage | 262 | 336 | 23104 | 12784 | 307 | 136 |
| 2nd Drips 1:47 | | | | | | |
| Second Evap Stage | 262 | 336 | 18439 | 10600 | 341 | 151 |
| 2nd Drips 1:36 | | | | | | |
| Radar Panel | 262 | 336 | 16991 | 10588 | 289 | 139 |
| (Condensate | | | | | | |
| composite) 1:33 | | | | | | |
| Thin Juice Softened | 394 | 478 | 56502 | −2 | 26175 | 55554 |
| Sulfur Caustic 2:05 pm | | | | | | |
| First Evap Stage | 262 | 336 | 40196 | 16692 | 275 | 132 |
| 2nd Drips 2:01 pm | | | | | | |
| Second Evap Stage | 316 | 384 | 42913 | 15157 | 285 | 110 |
| 2nd Drips 1:53 pm | | | | | | |
| RADAR Panel 1:57 pm | 316 | 384 | 30390 | 14810 | 268 | 129 |
| Boiler Radar DA 1:40 pm | 316 | 384 | 68334 | 10136 | 280 | 106 |
| Raff Radar 1:00 pm | 274 | 342 | 29793 | 28236 | 1614 | 725 |
| Raff Drips 1:07 pm | 272 | 328 | 9056 | 7320 | 180 | 74 |
| Thin Juice 1:22 pm | 396 | 488 | 43020 | 28 | 24799 | 41099 |
| Thin Juice Softened | 380 | 464 | 40684 | 90 | 33975 | 39360 |
| Sulfur Caustic 10:16 am | | | | | | |
| First Evap Stage | 314 | 382 | 12503 | 8504 | 404 | 227 |
| 2nd Drips 10:19 am | | | | | | |
| Second Evap Stage | 316 | 384 | 11997 | 7920 | 355 | 205 |
| 2nd Drips 10:24 am | | | | | | |
| RADAR Panel 10:27 am | 264 | 336 | 16736 | 10283 | 379 | 225 |
| Thin Juice Softened | 390 | 472 | 49806 | 12 | 30431 | 49771 |
| Sulfur Caustic 10:32 am | | | | | | |
| First Evap Stage | 262 | 336 | 29223 | 14735 | 366 | 177 |
| 2nd Drips 10:27 am | | | | | | |
| Second Evap Stage | 264 | 334 | 21153 | 12375 | 353 | 179 |
| 2nd Drips 10:29 am | | | | | | |
| RADAR Panel 10:19 am | 262 | 334 | 27051 | 13943 | 339 | 168 |
| DA Radar 12:37 pm | 270 | 336 | 7338 | 6364 | 233 | 96 |
| CSB Radar 2:20 pm | 268 | 338 | 16203 | 14776 | 956 | 351 |
| R1 Drips Raff 2:10 pm | 274 | 332 | 5859 | 5179 | 158 | 66 |
| Thin Juice 12:30 pm | 376 | 456 | 47630 | 64 | 40069 | 43639 |

TABLE 2

| | | | | Intensity | Intensity | Intensity | Lignin and | | | | |
|---|---|---|---|---|---|---|---|---|---|---|---|
| | Max | Max | Relative | at 275 nm, | at 365 nm, | at 390 nm, | Tannin | | | Max | Max |
| Sample | Excitation | Emission | Intensity | 350 nm | 470nm | 470 nm | Values | TOC | Sample | Excitation | Emission |
| Type | (nm) | (nm) | (A.U.) | (A.U.) | (A.U.) | (A.U.) | (ppm) | (ppm) | Type | (nm) | (nm) |
| Evaporator | 426 | 516 | 5420 | 0 | 200 | 1373 | Interference | 63510 | Evaporator | 426 | 516 |
| Supply | | | | | | | | | Supply | | |
| Juice | | | | | | | | | Juice | | |
| 1st Evap | 382 | 354 | 400 | 379 | 63 | 56 | 0.01 | 1.1 | 1st Evap | 382 | 354 |
| Condensate | | | | | | | | | Condensate | | |
| 2nd Evap | 264 | 332 | 44573 | 20294 | 1097 | 576 | 4.4 | 5.1 | 2nd Evap | 264 | 332 |
| Condensate | | | | | | | | | Condensate | | |
| Processing | 304 | 426 | 1372 | 747 | 806 | 525 | 0.9 | 2.6 | Processing | 304 | 426 |
| Well Water | | | | | | | | | Well Water | | |
| Processing | 310 | 430 | 1328 | 924 | 806 | 519 | 0.3 | — | Processing | 310 | 430 |
| Well Water | | | | | | | | | Well Water | | |
| Processing | 310 | 422 | 1300 | 608 | 684 | 512 | 0.3 | — | Processing | 310 | 422 |
| Well Water | | | | | | | | | Well Water | | |
| Filtered | | | | | | | | | Filtered | | |
| Water | 264 | 332 | 53719 | 27584 | 804 | 417 | — | — | Water | 264 | 332 |
| Water | 264 | 330 | 24101 | 11704 | 701 | 368 | — | — | Water | 264 | 330 |
| Water | 274 | 334 | 111322 | 88645 | 2617 | 805 | — | — | Water | 274 | 334 |
| Water | 274 | 334 | 78575 | 62187 | 1671 | 536 | — | — | Water | 274 | 334 |
| Condensate | 266 | 330 | 45600 | 24378 | 303 | 152 | 2.3 | — | Condensate | 266 | 330 |
| Pan #2 | | | | | | | | | Pan #2 | | |
| Condensate | 262 | 332 | 23988 | 9265 | 195 | 97 | 1.1 | — | Condensate | 262 | 332 |
| Condensate | 262 | 332 | 15498 | 5849 | 103 | 56 | 0.3 | — | Condensate | 262 | 332 |
| Condensate | 270 | 298 | 36652 | 6295 | 245 | 119 | 1.3 | — | Condensate | 270 | 298 |
| 1st Effect | | | | | | | | | 1st Effect | | |
| Condensate | 280 | 330 | 501261 | 342953 | 5806 | 1146 | 4.7 | — | Condensate | 280 | 330 |
| 2nd Effect | | | | | | | | | 2nd Effect | | |
| Evaporator 1 | 272 | 300 | 43161 | 9975 | 296 | 97 | 1.4 | 17 | Evaporator 1 | 272 | 300 |
| Condenser | 262 | 334 | 141602 | 47907 | 445 | 225 | 3.4 | 447 | Condenser | 262 | 334 |
| Condensate | 262 | 334 | 49196 | 29325 | 1275 | 670 | 5.2 | 420 | Condensate | 262 | 334 |
| 2nd Tank | | | | | | | | | 2nd Tank | | |
| Dryer | 264 | 332 | 37058 | 21496 | 601 | 379 | 1.8 | 253 | Dryer | 264 | 332 |
| Pan 4 | 280 | 336 | 89733 | 70085 | 967 | 602 | 1.9 | 122 | Pan 4 | 280 | 336 |
| Good | 270 | 330 | 349 | 243 | 12 | 9 | 0.2 | 6.6 | Good | 270 | 330 |
| Condensate | | | | | | | | | Condensate | | |
| Bad | 262 | 336 | 1464 | 612 | 27 | 22 | 0.3 | 33 | Bad | 262 | 336 |
| Condensate | | | | | | | | | Condensate | | |
| Syrup before | 436 | 528 | 24215 | −5 | 10102 | 15969 | 85 | INT | Syrup | 436 | 528 |
| decoloring | | | | | | | | | before | | |
| | | | | | | | | | decoloring | | |
| Syrup after | 368 | 440 | 55020 | 2979 | 44237 | 44404 | 45 | INT | Syrup after | 368 | 440 |
| decoloring | | | | | | | | | decoloring | | |
| Condensate | 274 | 326 | 1193 | 896 | 134 | 75 | 1.8 | 7.8 | Condensate | 274 | 326 |
| Clean | | | | | | | | | Clean | | |
| Condensate | 278 | 320 | 881 | 707 | 157 | 93 | 1.7 | 61 | Condensate | 278 | 320 |
| 0.1% sugar | | | | | | | | | 0.1% sugar | | |

Table 3 below shows data of samples taken from the thin juice of a U.S. sugar beet factory over the course of a campaign. The data shows an average max excitation wavelength of 368 nm and an average maximum emission wavelength of 467 nm.

TABLE 3

| Date of | Max | Max | Relative | |
|---|---|---|---|---|
| Sample | Excitation | Emission | Intensity | pH |
| Sep. 25, 2020 | 394 | 468 | 65901 | 10.89 |
| Sep. 28, 2020 | 356 | 440 | 46571 | 7.57 |
| Oct. 28, 2020 | 386 | 464 | 52593 | 8.44 |
| Sep. 9, 2020 | 374 | 454 | 44055 | 7.35 |
| Sep. 30, 2020 | 388 | 468 | 42696 | 8.5 |
| Dec. 7, 2020 | 394 | 478 | 56502 | 9.13 |
| Dec. 21, 2020 | 380 | 464 | 40684 | 6.26 |
| Jan. 4, 2021 | 390 | 472 | 49806 | 8.42 |
| Jan. 19, 2021 | 398 | 474 | 43258 | 8.6 |
| Feb. 2, 2021 | 392 | 466 | 50894 | 6.01 |
| Feb. 16, 2021 | 398 | 486 | 35416 | 8.95 |

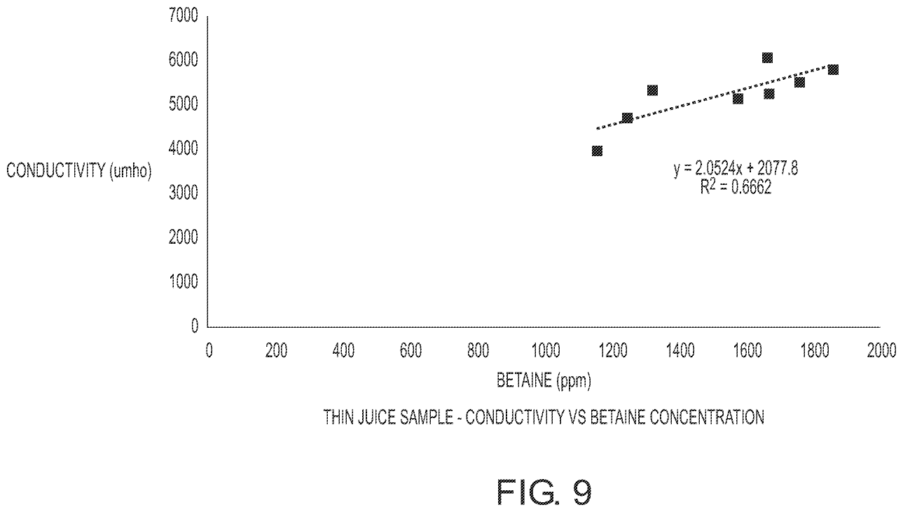

FIGS. 7-12 show various correlations of data taken from a U.S. sugar beet factory. FIG. 7 is a graph showing the correlation between the measured amount of sucrose in the thin juice and the measured fluorescence intensity of the thin juice from four fluorescence probes—(1) radar probe, 365 nm ex/470 nm em; (2) 316 nm ex/384 nm em; (3) 274 nm ex/350 nm em; and (4) 390 nm ex/470 nm em. The fluorescence intensity at 274/350 shows a good correlation (about 65%) to the amount of sucrose in the in the thin juice.

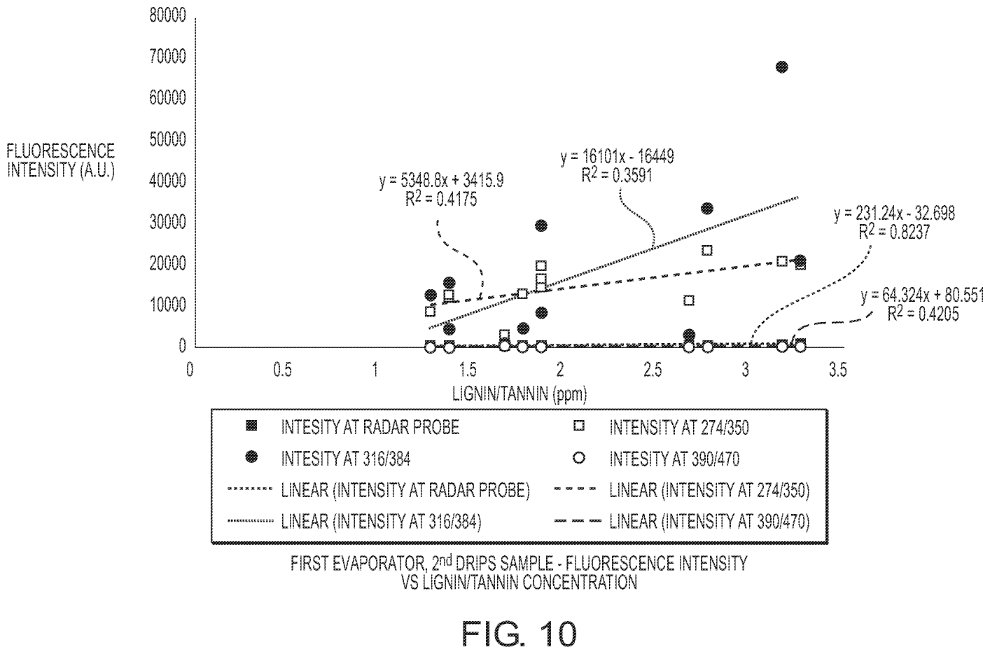

FIG. 8 is a graph that shows the correlation of the sucrose concentration in the thin juice to the amount of lignins/tannins in the thin juice. FIG. 8 shows that the concentration of concentration of lignins/tannins has a good correlation with the concentration of sucrose (about 69%).

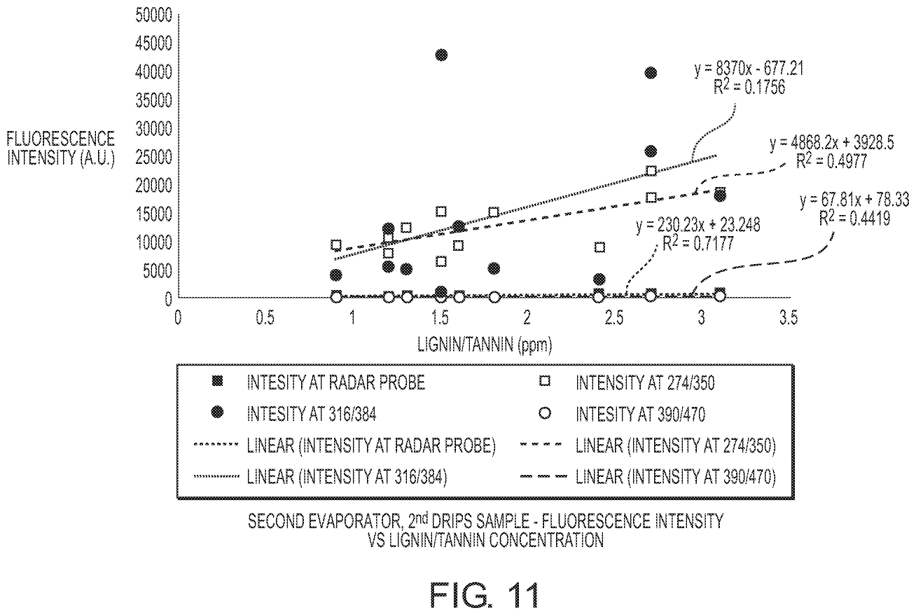

FIG. 9 is a graph that shows the correlation of the betaine concentration in the thin juice to the measured conductivity of the thin juice. The betaine concentration exhibits a good correlation with the conductivity (about 67%).

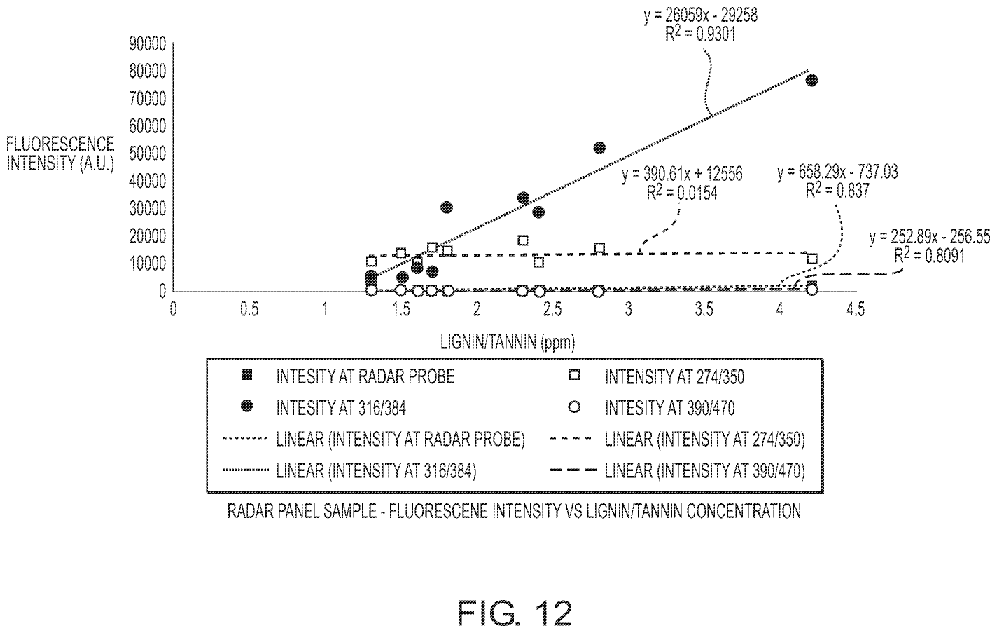

FIG. 10 is a graph showing the correlation between the measured amount of lignins/tannins in the condensate of a first evaporator and the measured fluorescence intensity at the wavelengths of the four probes identified above. The fluorescence intensity of the radar probe (365/470) shows a good correlation (about 82%) to the amount of lignins/tannins in this condensate stream.

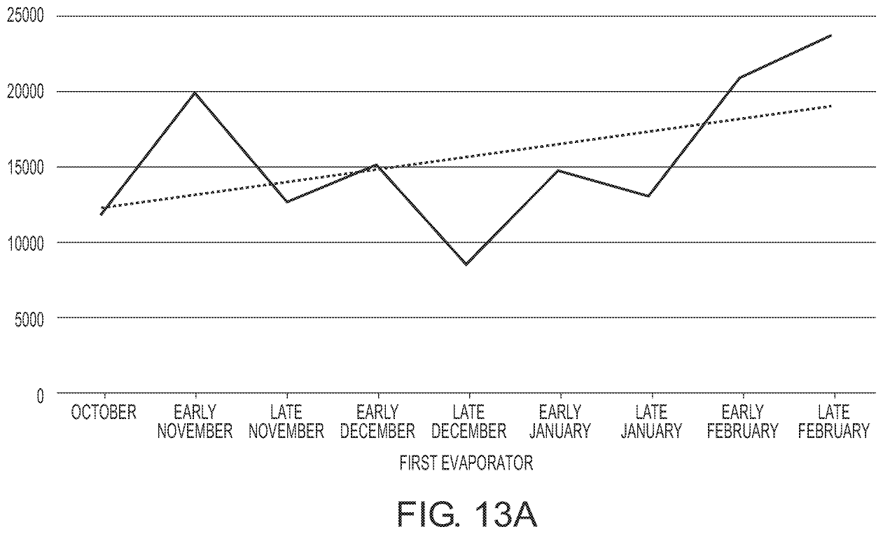

FIG. 11 is a graph showing the correlation between the measured amount of lignins/tannins in the condensate of a second evaporator and the measured fluorescence intensity at the wavelengths of the four probes identified above. The fluorescence intensity of the radar probe (365/470) shows a good correlation (about 72%) to the amount of lignins/tannins in this condensate stream.

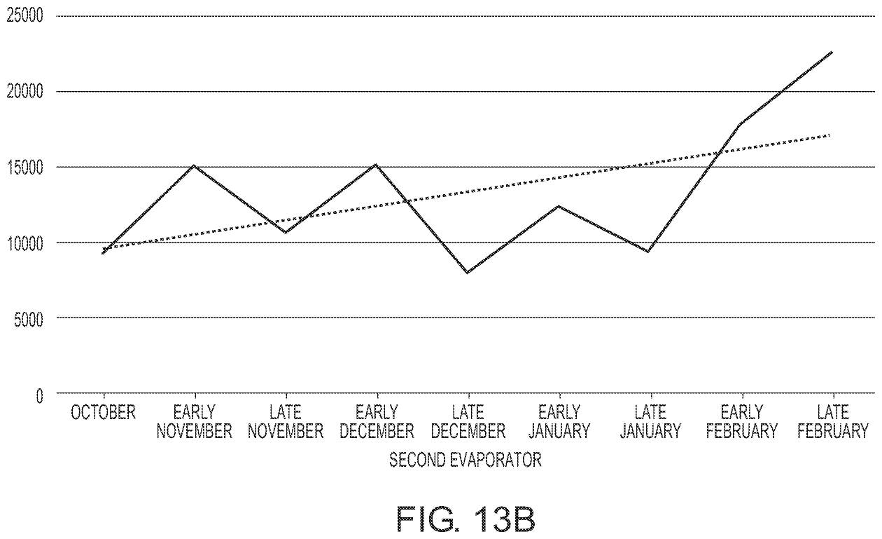

FIG. 12 is a graph showing the correlation between the measured amount of lignins/tannins in a radar panel sample and the measured fluorescence intensity at the wavelengths of the four probes identified above. The radar panel is located on the boiler feedwater. The fluorescence intensity of the radar probe, 316/384, and 390/470 all show good correlations, respectively at about 84%, 93%, and 81%.

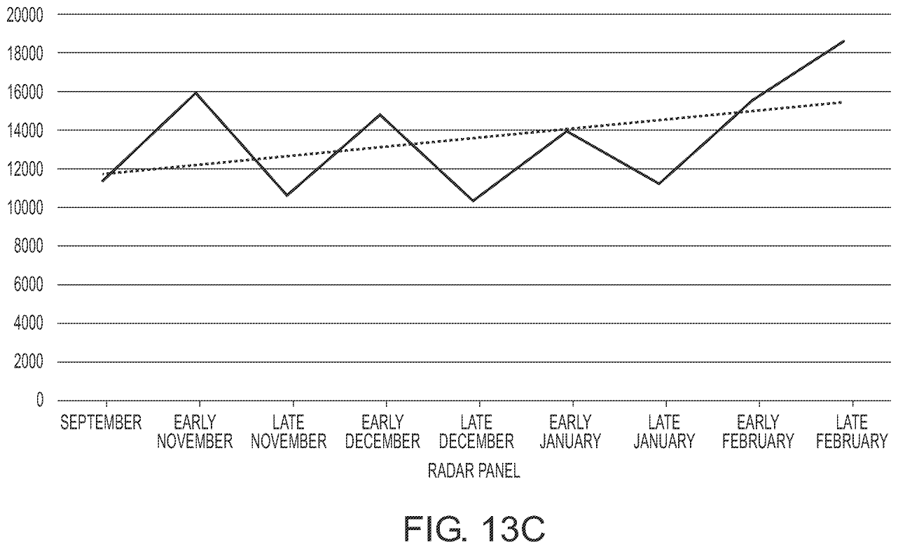

FIGS. 13A-13C are graphs illustrating the measured fluorescence intensity (275/350) at various stages in a sugar beet processing facility over the course of a campaign. FIG. 13A shows the measured fluorescence intensity of the condensate of the first evaporator, FIG. 13B shows the measured fluorescence intensity of the condensate of the second evaporator, and FIG. 13C shows the measured fluorescence intensity of the boiler feedwater at the radar panel. The graphs illustrate that the fluorescence intensity increases as the campaign progresses, which likely indicates that the concentration of contaminants and lower molecular weight components increases as the beets degrade if purification measures are not increased.

The parameters of fluorescence, conductivity, pH, TOC/ORP, or a combination thereof can be used to identify, quantify, track, and/or ultimately control those contaminates. In one aspect, these metrics can be used to control operating parameters, such as flow rate, pH, temperature, chemical addition, etc., at various stages to reduce the amount of contaminants in water sources that are used for the boiler feedwater. For example, the carb or lime steps identified above can be changed based on measured values, e.g., by feeding less or more coagulant, based on measured parameters. Likewise, since abrupt changes in one or more of the parameters can indicate spikes in contaminant levels, an operator can evaluate such changes and take corrective actions when necessary, such as adding a base to the water to mitigate pH drops and prevent the feedwater from becoming corrosive, using a different feedwater source (e.g., a different condensate drip), or taking the boiler off-line. Similarly, purification steps can be performed or increased on the boiler feedwater, feedwater source, or thin juice to reduce the overall concentration of contaminant.

These corrective actions can be taken if one or more of the parameters exceeds threshold values or are outside of preset target ranges. These operations can be automatic by using a processor that is programmed with control software, and inputs signals from the fluorescence probe, conductivity probe, ORP probe, and/or pH probe, determines whether a contamination event has occurred (e.g., if one or more signals exceeds a predetermined threshold, or changes at a predetermined rate), and optionally outputs control signals to control process equipment to correct the contamination event. Additionally, if the processor determines that a contamination event has occurred, it can issue a signal to display an alert or warning to the operator (e.g., on a displayed control dashboard) so that the operator can determine if corrective action should be taken.

Additionally, evaluating the above-identified parameters at a given facility could, over time, enable operators to predict when the presence of contaminants in water is likely to occur, e.g., based on the time of season, temperature, or process conditions. Accordingly, preventive measures could be taken in advance to limit the amount of contaminants that are likely to enter the boiler feedwater.

It will be appreciated that the above-disclosed features and functions, or alternatives thereof, may be desirably combined into different systems or methods. Also, various alternatives, modifications, variations or improvements may be subsequently made by those skilled in the art, and are also intended to be encompassed by the disclosed embodiments. As such, various changes may be made without departing from the spirit and scope of this disclosure.

What is claimed is:

1. A method for evaluating water that is used as boiler feedwater, the method comprising:
measuring at least one parameter of the water that is selected from the group consisting of pH, conductivity, total organic carbon (TOC), and oxidation reduction potential (ORP); and
based on the at least one measured parameter, determining whether to take corrective action to reduce an amount of organic contaminant in the water and/or mitigate effects of the organic contaminant in the water.

2. The method according to claim 1, the measuring comprising measuring at least one of the pH and the conductivity of the water.

3. The method according to claim 1, further comprising measuring a fluorescence intensity of the water, and determining whether to take the corrective action based on the measured fluorescence intensity.

4. The method according to claim 1, further comprising taking the corrective action when it is determined that the at least one measured parameter changes at a rate that exceeds a threshold value.

5. The method according to claim 3, further comprising taking the corrective action when it is determined that the at least one measured parameter and the fluorescence intensity changes at a rate that exceeds a threshold value.

6. The method according to claim 1, further comprising taking the corrective action when it is determined that the at least one measured parameter exceeds a threshold value.

7. The method according to claim 1, wherein the boiler feedwater includes condensate from an evaporation process.

8. The method according to claim 7, wherein water that is measured includes the condensate.

9. The method of claim 1, further comprising measuring a concentration of at least one organic contaminant in the water and correlating the measured concentration to the at least one measured parameter.

10. The method of claim 9 wherein the measured organic contaminant includes lignins and tannins.

11. The method of claim 1, the measuring comprising measuring the conductivity of the water, and further comprising measuring a fluorescence intensity of the water, and determining whether to take the corrective action based on the measured conductivity and the measured fluorescence intensity.

12. The method of claim 3, wherein measuring the fluorescence intensity includes measuring emission intensity at a wavelength in a range of 380-400 nm.

13. The method of claim 3, wherein measuring the fluorescence intensity includes measuring emission intensity at a wavelength in a range of 340-360 nm.

14. The method of claim 9, wherein the organic contaminant is at least one selected the group consisting of sugars, lignins, tannins, organic acids, and a breakdown product of these compounds.

15. The method of claim 9, wherein the organic contaminant includes at least one of lignins and tannins.

16. An apparatus for evaluating water that is used as boiler feedwater in a food processing facility, the apparatus includes a processor that is programmed to:
receive a signal corresponding to at least one measured parameter of the water that is selected from the group consisting of pH, conductivity, total organic carbon (TOC), and oxidation reduction potential (ORP); and
based on the received signal corresponding to the at least one measured parameter, generating a signal to control at least one operating parameter of the food processing facility and/or generating a signal that causes a display to display an alert.

17. A method for controlling an amount of an organic contaminant in boiler feedwater used in a boiler of an evaporator stage in a sugar processing facility, the method comprising:
measuring at least one parameter of the boiler feedwater that is selected from the group consisting of pH, conductivity, total organic carbon (TOC), and oxidation reduction potential (ORP); and
based on the at least one measured parameter, taking at least one corrective action to reduce an amount of organic contaminant in the boiler feedwater and/or mitigate effects of the organic contaminant in the boiler feedwater.

18. The method of claim 17, wherein the at least one corrective action includes changing an operating parameter of a stream in the sugar processing facility, including at least one of flow rate, pH, temperature, and addition of chemicals to the stream.

19. The method of claim 17, wherein the at least one corrective action includes changing a feedwater source to the boiler.

20. The method of claim 17, wherein the at least one corrective action includes taking the boiler offline.
